(19) United States
(12) Plant Patent Application Publication
(10) Pub. No.: US 2014/0137302 P1
(43) Pub. Date: May 15, 2014

(54) Vaccinium hybrid plant named 'RIDLEY 1403'

(50) ;

(71) Applicant: MOUNTAIN BLUE ORCHARDS PTY LTD., Lindendale (AU)

(72) Inventor: Ridley Bell, Lindendale (AU)

(73) Assignee: MOUNTAIN BLUE ORCHARDS PTY LTD., Lindendale (AU)

(21) Appl. No.: 13/694,258

(22) Filed: Nov. 13, 2012

(51) Int. Cl. A01H 5/00

(52) U.S. Cl. PLT/157

(57) ABSTRACT

A new and distinct cultivar of Vaccinium hybrid plant named “RIDLEY 1403”, characterized by its combination of upright to semi-upright plant growth habit and strong plant growth vigor, evergreen foliage, very early flowering, early to medium time of ripening of fruit, very large fruit size, medium firm fruit suited to handling, attractive dark blue fruit color, flavorsome fruit with good sweetness and acidity balance and a small dry picking scar. This combination results in high quality fruit with larger size than other varieties.

### LATIN NAME OF GENUS AND SPECIES OF PLANT CLAIMED

Vaccinium hybrid

### VARIETY DENOMINATION

“RIDLEY 1403”

### BACKGROUND OF THE INVENTION

The present invention relates to a new and distinct perennial variety of Vaccinium hybrid (Southern Highbush Blueberry), which has been given the variety denomination of “RIDLEY 1403”. Its market class is that of a fruiting plant. “RIDLEY 1403” is intended for use as fresh fruit for shipping, customer pick and processing markets and as a home garden plant.

The new Vaccinium hybrid cultivar is a selection resulting from seedlings produced in a breeding programme of Vaccinium at Lindendale, NSW, Australia in 2006 from a controlled pollination of seed parent “S02-25-05” (unpatented) by pollen parent “S03-08-02” (unpatented). The new cultivar was discovered and selected as a single plant designated “M08-14-03” within a population of one hundred plants of Vaccinium hybrids in 2008 in a commercial field plantation environment at Lindendale, New South Wales, Australia. Selection criteria was a combination of strong plant growth vigor, low chilling requirement, early to medium season of fruit ripening, small picking scar, strong berry firmness, high yield, very large berry size, good flavor, and sweet, crisp fruit. The selection was subsequently evaluated for over two years at commercial farms at Lindendale, New South Wales, Australia.

Asexual reproduction of the new cultivar by cutting propagation since 2008 at Lindendale, New South Wales, Australia and has demonstrated that the new cultivar reproduces true to type with all of the characteristics, as herein described, firmly fixed and retained through successive generations of such asexual propagation.

The new variety has been primarily compared to the variety “C99-42” (U.S. patent application Ser. No. 12/228,525). In Lindendale, New South Wales, Australia the plant growth vigour of “RIDLEY 1403” is ‘strong’ whereas the plant growth vigour of “C99-42” is ‘medium to strong’. The leaf width of “RIDLEY 1403” is ‘broad’ (mean 35.1 mm) whereas the leaf width of “C99-42” is ‘medium to broad’ (mean 30.2 mm). The berry diameter of “RIDLEY 1403” is ‘very large (mean 24.0 mm) whereas the berry diameter of “C99-42” is ‘large’ (mean 18.4 mm). The berry calyx basin diameter of “RIDLEY 1403” is ‘large’ (mean 8.1 mm) whereas the berry calyx basin diameter of “C99-42” is ‘medium’ (mean 6.3 mm). The berry sweetness of “RIDLEY 1403” is rated as ‘high’ whereas the sweetness of “C99-42” is ‘medium’. The berry acidity of “RIDLEY 1403” is rated as ‘medium to high’ whereas the acidity of “C99-42” is ‘low to medium’. This combination of vegetative and fruiting attributes results in “RIDLEY 1403” being a desirable variety for mid season commercial production.

“RIDLEY 1403” can also be compared to the variety “C03-158” (unpatented). The plant growth habit of “RIDLEY 1403” is ‘upright to semi-upright’ whereas the plant growth habit of “C03-158” is ‘semi-upright’. In Lindendale, New South Wales, Australia the time of vegetative bud burst of “RIDLEY 1403” is classified as ‘early to medium’ whereas the time of vegetative bud burst of “C03-158” is classified as ‘early’. The time of beginning of flowering of “RIDLEY 1403” is classified as ‘very early’ whereas the time of beginning of flowering of time of “C03-158” is classified as ‘early to medium’. The berry shape of “RIDLEY 1403” is round whereas the berry shape of “C03-158” is oblate. The berry diameter of “RIDLEY 1403” is ‘very large (mean 24.0 mm) whereas the berry diameter of “C03-158” is ‘large’ (mean 18.5 mm).The berry calyx basin diameter of “RIDLEY 1403” is ‘large’ (mean 8.1 mm) whereas the berry calyx basin diameter of “C03-158” is ‘medium to large’ (mean 7.2 mm). The depth of berry calyx basin of “RIDLEY 1403” is ‘deep’ whereas the depth of berry calyx basin of “C03-158” is ‘shallow’. The berry acidity of “RIDLEY 1403” is ‘medium to high’ whereas “C03-158” is ‘medium’. See Table 1 for comparisons

### SUMMARY OF THE INVENTION

The following characteristics of the new cultivar have been repeatedly observed and can be used to distinguish “RIDLEY 1403” as a new and distinct cultivar of Vaccinium hybrid plant:

1. Upright to semi-upright plant growth habit

2. Very early timing of beginning of flowering

3. Early-medium timing of fruit ripening

4. Strong plant growth vigor

5. Berry diameter is very large

6. Dark blue berry color

7. Berry firmness is medium

8. Berry sweetness is medium

9. Berry acidity is medium to high

10. Picking scar is dry

Plants of the new cultivar differ from plants of the seed parent “S02-25-05” in that the berry diameter of “RIDLEY 1403” is ‘very large whereas the berry diameter of “S02-25-05” is medium. Plant growth vigour of “RIDLEY 1403” is ‘strong’ whereas the plant growth vigour of the seed parent is medium. Berry color is very dark whereas the seed parent berry color is medium dark. Plants of “Ridley 1403” have an upright to semi-upright plant growth habit which is less bushy than plants of the seed parent which have a bushy semi-upright growth habit. Plants of the new cultivar differ from plants of the pollen parent “S03-08-02” in that the berry diameter of “RIDLEY 1403” is ‘very large whereas the berry diameter of “S03-08-02” is medium. Plants of “Ridley 1403” have an upright to semi-upright plant growth habit which is less bushy than plants of the pollen parent which have a very bushy semi-upright growth habit. The berry sweetness of “RIDLEY 1403” is rated as ‘high’ whereas the sweetness of the pollen parent is ‘medium’.

### BRIEF DESCRIPTION OF THE PHOTOGRAPHS

The accompanying photographs show, as nearly true as it is reasonably possible to make the same in color illustrations of this type, typical flower and foliage characteristics of the new cultivar. Colors in the photographs differ slightly from the color values cited in the detailed description, which accurately describes the colors of “RIDLEY 1403”.

FIG. 1 illustrates a “RIDLEY 1403” in a commercial field planting.

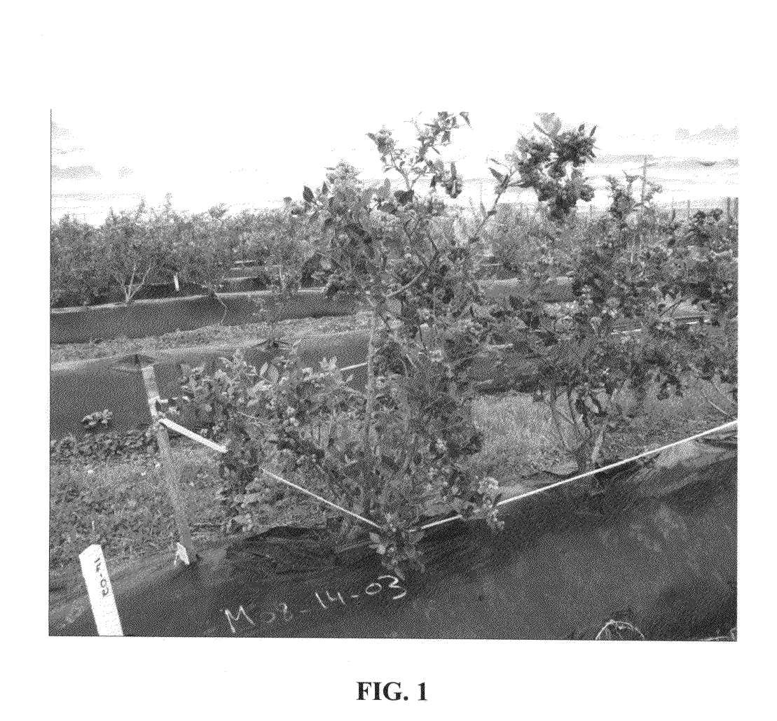

FIG. 2 illustrates a “RIDLEY 1403” leaf, flower and berry shapes and dimensions.

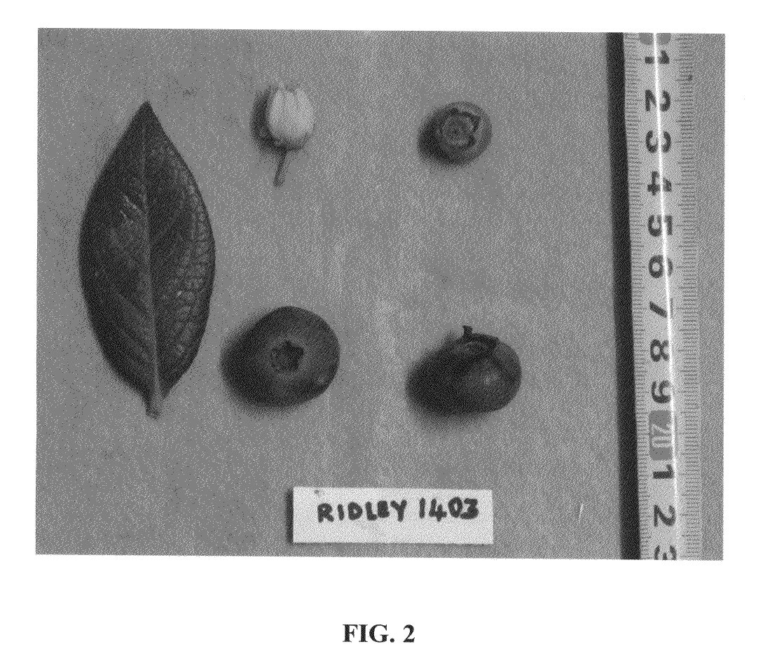

FIG. 3 illustrates “RIDLEY 1403” berries

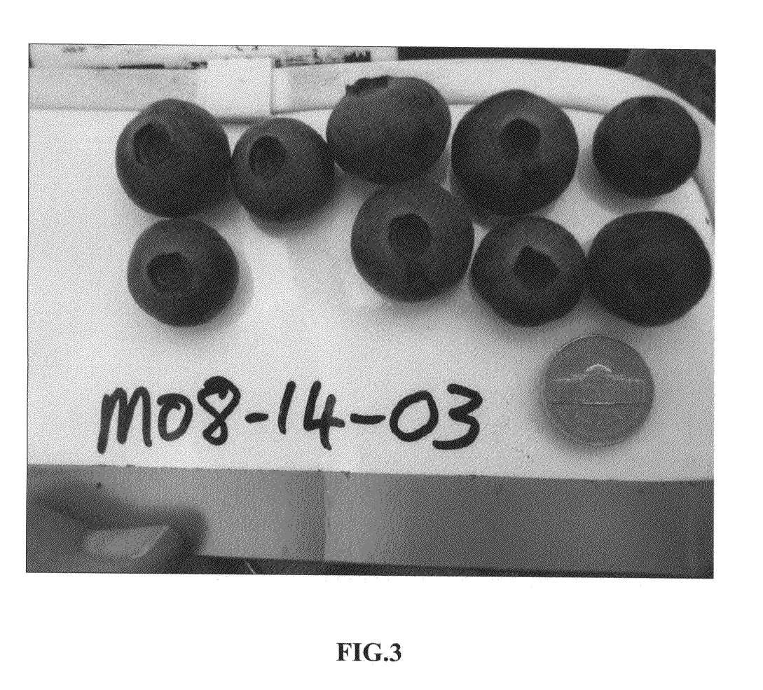

FIG. 4 illustrates a “RIDLEY 1403” flowers

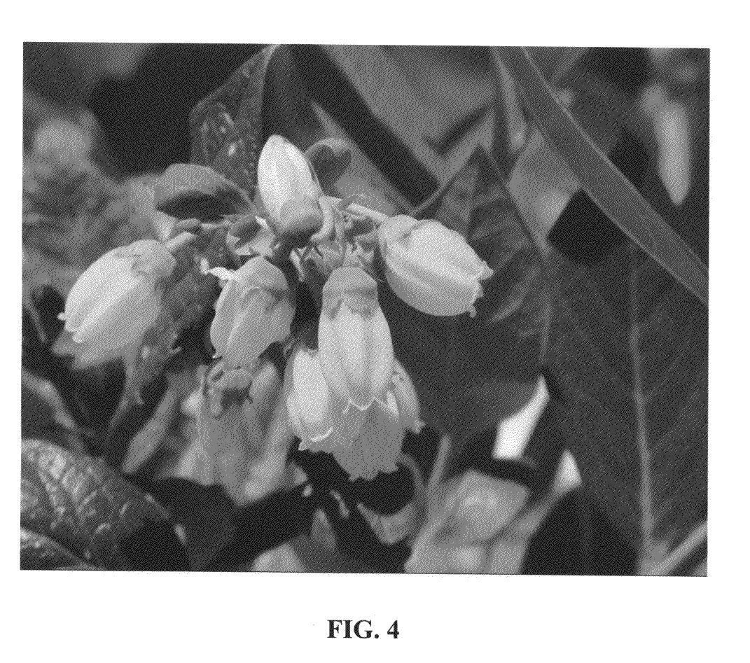

### DETAILED BOTANICAL DESCRIPTION

The following is a detailed botanical description of a new and distinct variety of a Vaccinium hybrid ornamental plant known as “RIDLEY 1403”. Plant observations were made on plants grown in Lindendale, New South Wales, Australia. Unless indicated otherwise, the descriptions disclosed herein are based upon observations made in August 2009 of mature “RIDLEY 1403” plants (age 4 years) grown in outdoor field plantings with day temperature ranging from 21° C. to 23° C., night temperatures ranging from 10° C. to 12° C., and light levels ranging from 6 to 8 klux. Those skilled in the art will appreciate that certain characteristics will vary with older or, conversely, younger plants. “RIDLEY 1403” has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light quality, light intensity, day length, cultural conditions and the like.

Color notations are based on The Royal Horticultural Society Color Chart, of The Royal Horticultural Society, London, 2007 edition.
Botanical classification: Vaccinium hybrid cultivar “RIDLEY 1403”.
Parentage: Parents — seed parent “S02-25-05” (unpatented) — pollen parent “S03-08-02” (unpatented)
Plant:
   Growth habit.—upright to semi-upright. Growth vigor is strong. Plant height is approximately 2.0 m. Plant width is approximately 1.2 m. Flower bud density is medium. Twigginess is low. Time of beginning of flowering is very early (begins late April to early May; peaks mid July) when grown as an evergreen in Australia. Time of fruit ripening is early to medium (begins mid July; peaks mid October) when grown as an evergreen in Australia.
Stem:
   Suckering tendency.—Plants typically have 5-7 major canes per plant from a base 25 cm in diameter on 4 year old plants.
   Canes.—erect to semi-erect with moderate branching; color greyed orange 198C; 2 year old cane diameter 11 mm, colored grayed orange 177B, some flaking of bark, current season wood diameter 3-4 mm, colored yellow green 144C Internode length on strong, new shoots 15-20 mm.
   Fruiting wood.—numerous twigs to 10 cm in length.
   Surface texture of new wood.—smooth.
Fully developed leaf: Length long 70-80 mm Width broad approximately 35 mm Shape elliptic Color yellow green 137A Intensity of color medium Margin entire Undulation of margin weak Pubescence of upper side absent Pubescence of lower side absent Cross sectional profile flat Longitudinal profile straight Attitude broad acute to horizontal
Inflorescence: Length of peduncle to 12 mm Length of pedicel long 4-6 mm Number of flowers per cluster median 8 Flower cluster density is medium-dense
Flowers: Length of corolla tube medium to long (to 16 mm) Width of corolla tube medium to broad (to 10 mm) Corolla aperture diameter 3-5 mm Color of corolla white 155D Anthocyanin coloration of corolla absent Corolla ridges present Protusion of stigma absent Fragrance weak Shape urceolate Abundance of pollen high Calyx (with sepals) diameter 5-6 mm Calyx color green 138A
Productivity: 3 to 4 kg per season from 3-4 year old plants planted at 3.0 m×0.9 m density.
Cold hardiness: “RIDLEY 1403” has not been grown in all environments including harsh winter environments. Cold tolerance is expected to be low
Chilling requirement: “RIDLEY 1403” has not been grown in all environments and is typically grown as an evergreen crop where chilling hours are not important. “RIDLEY 1403” is classed as ‘low chill’, typical of Southern Highbush Blueberry varieties with an estimated chilling requirement of 250 hours (not tested).
Leafing: plants retain leaves year round (at Lindendale, NSW Australia) and “RIDLEY 1403” is grown commercially as an evergreen crop. New leafing occurs in mid to late August at Lindendale, NSW Australia.
Fruit: Unripe fruit color intensity light Unripe fruit color 144A Cluster density is medium to dense. Diameter is very large (mean 24 mm) Weight heavy (approximately 5.2 g) Shape round Attitude of calyx erect Diameter of calyx basin large (mean 8.1 mm) Depth of calyx basin is deep (to 3.0 mm) Intensity of bloom medium Intensity of blue color of skin with bloom removed is dark Color of skin 202A Sweetness when ripe medium Firmness when ripe medium Acidity when ripe medium to high Size of scar is small (1.0-1.5 mm) Storage quality good
Date of 50% maturity: approximately 10^{th} October at Lindendale, NSW Australia.
Fruit development period: approximately 75 days in protected cultivation at Lindendale, NSW Australia.
Seed: Seed color greyed orange 165B Seed abundance in fruit medium Seed size 1.6-1.8 mm long for fully developed seeds

TABLE 1
Comparison with other varieties (extract from Australian PBR data)

| Organ/Plant | Ridley | | |
|---|---|---|---|
| Part: Context | 1403 | C99-42 | C03-158 |
| Plant: growth | strong | medium | medium |
| vigour | | to strong | to strong |
| Plant: growth | upright to | upright to | semi upright |
| habit | semi upright | semi upright | |
| Leaf: length | long to very | long to very | long to very |
| | long | long | long |
| Leaf: width | broad | medium to | broad |
| | | broad | |
| Leaf: shape | elliptic | elliptic | elliptic |
| Leaf: colour of | green | green | green |
| upper side | | | |
| Leaf: intensity | medium | dark | light |
| of green colour | | | |
| on upper side | | | |
| (varieties with green | | | |
| leaf colour only) | | | |
| Leaf: margin | entire | entire | entire |
| Flower: size of | medium to | medium | medium |
| corolla tube | large | | |
| Flower: anthocyanin | absent or | weak- | absent or |
| coloration of | very weak | medium | very weak |
| corolla tube | | | |
| Flower: ridges on | present | present | present |
| corolla tube | | | |
| Flower: protusion | absent | present | absent |
| of stigma | | | |
| Fruit cluster: density | medium | dense | medium |
| | to dense | | |
| Unripe fruit: intensity | light | light | light |
| of green colour | | | |
| Fruit: size | very large | large | large |
| Fruit: shape in | round | round | oblate |
| longitudinal section | | | |
| Fruit: attitude of sepals | erect | erect | erect |
| Fruit: diameter of | large | medium | medium to |
| calyx basin | | | large |
| Fruit: depth of calyx | deep | deep to | shallow |
| basin | | very deep | |
| Fruit: intensity of bloom | medium | medium | medium |
| Fruit: colour of skin | dark blue | dark blue | dark blue |
| Fruit: firmness | medium | firm | medium to |
| | | medium | firm |
| Fruit: sweetness | medium | medium | low to |
| | | | medium |
| Fruit: acidity | medium to | low to | medium |
| | high | medium | |
| Fruit: size of scar | small | small | small |
| Fruit: average weight | 5.2 | 2.4 | 2.8 |
| of ripe berry (g) | | | |
| Time of: vegetative | early to | medium | early |
| bud burst | medium | to late | |
| Time of: beginning of | very early | early to | early to |
| flowering on | | medium | medium |
| current years shoot | | | |
| Time of: beginning of | early to | early to | early to |
| fruit ripening | medium | medium | medium |
| on current years shoot | | | |

What is claimed is:

1. A new and distinct cultivar of Vaccinium hybrid plant named “RIDLEY 1403”, substantially as herein shown and described.
